(19) United States
(12) Patent Application Publication
(10) Pub. No.: US 2025/0186454 A1
(43) Pub. Date: Jun. 12, 2025

(54) METHODS FOR TREATING ABERRANT BEHAVIOR AND MOTOR ACTIVITY

(71) Applicant: LAPKO INC, dba AFECTA PHARMACEUTICALS, Las Vegas, NV (US)

(72) Inventor: Bruce KOVACS, Long Beach, CA (US)

(21) Appl. No.: 19/062,916

(22) Filed: Feb. 25, 2025

Related U.S. Application Data
(63) Continuation of application No. PCT/US2024/020271, filed on Mar. 15, 2024.
(60) Provisional application No. 63/491,029, filed on Mar. 17, 2023.

(51) Int. Cl. A61K 31/5377; A61K 9/00; A61P 25/00

(52) U.S. Cl. CPC A61K 31/5377; A61K 9/006; A61P 25/00

(57) ABSTRACT

Administration of molindone for the treatment of the aberrant behavior and motor activity symptoms associated with autism spectrum disorders (ADS), also found in subjects diagnosed with Fragile X, tuberous sclerosis, Smith-Lemli-Opitz syndrome, and maternally-inherited duplications of the Prader-Willi/Angelman syndrome region, is disclosed.

### CROSS-REFERENCE TO RELATED APPLICATIONS

This application is a Continuation application of PCT International Application No. PCT/US2024/020,271 filed on Mar. 15, 2024, which claims priority under 35 USC § 119 of Application No. 63/491,029 filed in the United States of America on Mar. 17, 2023, the entire contents of each of which are hereby incorporated by reference.

### FIELD OF THE INVENTION

The present disclosure pertains to treatment of the aberrant behavior and motor activity symptoms associated with autism spectrum disorders (ADS), such as motor stereotypy, as well as treatment of those symptoms also found in subjects diagnosed with, for example, Fragile X, tuberous sclerosis, Smith-Lemli-Opitz syndrome, and maternally-inherited duplications of the Prader-Willi/Angelman syndrome region.

### BACKGROUND

Autism spectrum disorders (ASDs) represent a syndrome and refers to a specific group of childhood developmental disorders that all manifest as a constellation of symptoms which consistently occur together. The onset is generally before the age of 3 years, and ASD is usually first diagnosed in early childhood. ASDs can range from a severe form, called autistic disorder, through pervasive development disorder not otherwise specified (PDD-NOS), to a much milder form, commonly known as Asperger's syndrome. ASDs have a prevalence of approximately 0.6% in the population.

Autism spectrum disorders cause severe and ubiquitous impairment in thinking, feeling, behavior, language, and the ability to relate to others. Persons with ASDs typically exhibit aberrant patterns of behavior and motor activities, as manifested by stereotyped motor activity or repetitive motor movements, increased irritability, inflexible adherence to routines, ritualized patterns of verbal, non-verbal behavior, hyperactive behavior, social avoidance, and emotional lability. Thus, the disorder is comprised of both behavioral and motor symptoms that interfere with daily living.

According to the Diagnostic and Statistical Manual of Mental Disorders, one essential diagnostic feature of autistic spectrum disorders is the presence of restricted, repetitive, and stereotyped patterns of behaviors, stereotyped or repetitive motor, and interests (collectively referred to as stereotypy). Common examples of motor stereotypy are hand flapping, body rocking, toe walking, spinning objects, sniffing, immediate and delayed echolalia, and running objects across one's peripheral vision. The Diagnostic and Statistical Manual of Mental Disorders (DSM-IV) criteria for stereotypies requires repetitive, nonfunctioning behaviors that are present for greater than 4 weeks and that interfere with normal activities. Stereotypic behaviors are not restricted to autism. They are common to individuals with other sensory, intellectual, or developmental disabilities. For example, research indicates that a large majority of individuals with mental retardation exhibit stereotypies. However, in comparison to individuals with mental retardation, those with autism tend to display more varied topographies, along with increased severity and overall occurrence. Other essential features of ASDs are abnormal social communication and interpersonal interaction skills, inattentive behaviors, hyper-irritability, abnormal mood and/or emotional reactions to events, and persistent agitation.

The cause or causes of the most common form of ASD is unknown. However, some rare genetic syndromes and chromosomal anomalies explain roughly 10% of cases of autism, including Fragile X, tuberous sclerosis, Smith-Lemli-Opitz syndrome, and maternally-inherited duplications of the Prader-Willi/Angelman syndrome region (chromosome 15q11-13). However, despite high heritability, genetic studies to date have not provided substantial insight into most cases of ASD with idiopathic etiology. Emerging science suggests that circuit-level explanations of ASD pathogenesis are appealing because they most directly account for the emergence of clinical symptoms.

The persistent and pervasive behavior and motor symptoms associated with autism spectrum disorder are the most significant concerns reported by parents in early childhood. These symptoms persist into later childhood, adolescence, and early adulthood. They effect learning and socialization. There are no FDA approved medications to treat the motor and behavioral symptoms associated with autism spectrum disorders. In addition, no single drug or protocol of multiple drugs has been proven to be entirely satisfactory for addressing the behavioral and motor symptoms associated with ASD. Importantly, drugs currently used off-label display adverse side effects which limit their use, especially in children under the age of 13.

There is a continuing need in the art for compounds that can not only address the underlying causes of autism spectrum disorders but alleviate the associated behavioral abnormalities common to the associated conditions. Compounds with reduced toxicity profiles and good bioavailability are especially desirable since children are often the target of potential therapeutic agents.

### SUMMARY

The present disclosure relates to a method of treating one or more motor and/or behavioral symptoms in a subject with ASD. The method consists of topically administering an effective amount of molindone or a pharmaceutically acceptable salt thereof either as the racemate or as a specific enantiomer topically to the buccal, lingual, sublingual, nasal, or respiratory mucosa of the subject where one or more aberrant behavior(s) or motor symptom(s) of ASD are alleviated in the subject. Alternatively, the method consists of topically administering an effective amount of molindone or a pharmaceutically acceptable salt thereof either as the racemate or a specific enantiomer topically to the epidermis of the subject wherein one or more aberrant behavior(s) or motor symptom(s) of ASD are alleviated in the subject.

### BRIEF DESCRIPTION OF DRAWINGS

Table 1 shows the data resulting from a physician initiated study of molindone in patients with autism spectrum disorder using the ABC psychometric scale.

FIG. 1 shows the pharmacokinetic data of equivalent doses of molindone administered as a tablet to the stomach compared to administration by sublingual dose form.

### DETAILED DESCRIPTION

As used herein the terms “administer”, “administering” or “administration” refer to any method in medical practice that delivers a provided composition or active pharmaceutical composition(s) or biologically active agent to a subject in need of treatment thereof in a manner to produce a therapeutic, or ameliorative effect to the subject.

As used herein, the term “Autism Spectrum Disorders” (ASDs) or “Autism Spectrum Disorder” (ASD) in the singular, refers to a syndrome specific group of childhood developmental disorders that all manifest as a constellation of symptoms which consistently occur together. Diagnostic criteria of Autism Spectrum Disorders are found the Diagnostic and Statistical Manual of Mental Disorders, Fifth Edition (DSM-5) and encompass the previous DSM manual's autistic disorder (autism), Asperger's disorder, childhood disintegrative disorder, and pervasive developmental disorder not otherwise specified. As used herein, the term “Autism Spectrum Disorders” (ASDs) or the singular form ASD is used without regard to any known or unknown possible underlying causes or co-occurring disorders. ASDs are characterized by aberrant behaviors including, but not limited to, the following: deficits in social communication and social interaction as well as restricted repetitive behaviors, stereotypic motor activities, persistent and disproportional agitation, or irritability.

As used herein the term “Aberrant Behavior” refers to: irrational or exaggerated fear, social avoidance, compulsive behavior, manic activity, irritability, lethargy, persistent agitation, motor stereotypy, and inappropriate speech and/or any combination thereof. Aberrant behavior includes many of the symptoms present in ASDs and the Aberrant Behavior Checklist (ABC) and its subscales are useful in quantitative measurement of the severity of the aberrant behaviors manifested in a subject with ASD.

As used herein, “Aberrant Behavior Checklist” (ABC) is an informant rating instrument that was empirically derived by principal component analysis. It contains 58 items that resolve onto five subscales. The subscales and the respective number of items are as follows: (a) irritability (15 items), (b) lethargy/social withdrawal (16 items), (c) stereotypic behavior (7 items), (d) hyperactivity/noncompliance (16 items), and (e) inappropriate speech (4 items). The ABC is well recognized and accepted as a useful psychometric instrument for use in ASD.

As used herein the terms “stereotypy” and “stereotyped” refer to restricted, repetitive patterns of motor activity or movements and behaviors which often, but not always, seem purposeless.

As used herein, the term “syndrome” refers to a group of symptoms of disordered function related to one another by means of some anatomical, physiological, or biochemical peculiarity which does not include a precise cause of an illness.

As used herein, the term “symptom” refers to any perceptible change in the body or its functions that indicates disease or the kind or phases of disease. Aspects of general symptom analysis include the following: onset: date, manner (gradual or sudden), and precipitation factors; characteristics: character, location, radiation, severity, timing, aggravating, or relieving factors, and associated symptoms; course since onset: incidence, progress, and effects of therapy. A symptom or group of symptoms can occur in several etiologically different conditions, disorders, or diseases. A non-limiting example of this concept is fever and chills, two different symptoms which can occur in several unrelated disorders such as toxic chemical poisoning or appendicitis.

As used herein, the term “treating” or “treatment” refers to mitigating, improving, relieving, ameliorating, or alleviating at least one symptom (such as an aberrant behavior symptom, an aberrant motor activity symptom, or a combination thereof) of a condition, disease, or disorder in a human subject, or the improvement of an ascertainable measurement associated with a condition, disease, or disorder.

As used herein the term “mucosa” refers to the lining of most of the body cavities and hollow internal organs such as, but not limited to, the mouth, nose, eyelids, lungs, and the vagina. The term mucous membrane(s) is synonymous with the terms: mucosa, mucosal tissues, and mucosal epithelium. The mucosa tissues consist of an epithelial layer on a basement membrane and a connective tissue layer (the lamina propria) and contain goblet-shaped cells that secrete mucus which keeps the surface moist and lubricated.

As used herein the term “buccal mucosa” (also known as mucosa buccalis) refers to the inner lining of the cheeks and lips, which is an anatomic region that includes all the mucous membrane lining of the inner surface of the cheeks and lips, from the line of contact of the opposing lips to the line of attachment of mucosa to the alveolar ridges and pterygomandibular raphe.

As used herein the term “sublingual mucosa” refers to the area of the oral cavity that is underneath the tongue. For example, a sublingual medication is a medication that is designed to be dissolved under the tongue and absorbed directly through the sublingual mucosa without the need to swallow the medication and thereby precluding either gastric or intestinal absorption.

As used herein the term “lingual mucosa” refers to the mucosa forming the superior and inferior surface of the tongue; that of the superior surface (i.e., dorsum) of the tongue appears velvety due to the presence of vast numbers of papillae while that of the inferior surface is smooth and thinner.

As used herein the term “nasal mucosa” refers to mucous membranes that line the nares, nasal cavity, and nasopharynx.

As used herein the term “respiratory mucosa” refers to the respiratory epithelium found lining most of the respiratory tract as a pseudostratified or ciliated columnar epithelium composed of six cell types. Three of these, goblet cells, ciliated columnar cells, and basal cells, constitute a majority of the cell population.

As used herein the term “muco-adherent” is synonymous with the term “mucoadhesive” and refers to substances that stick to or adhere to the mucosal tissues or mucous membranes by forces that are measurable and by any number of mechanisms such as, but not limited to the following: hydrogen-bonding, ionic interaction, covalent bonding, hydrophobic interaction, Vander Waals interaction, or combinations thereof.

As used herein the term “penetration enhancer” is synonymous with the terms; “permeation enhancers”, and “absorption enhancers,” and refers to chemical compounds that can facilitate the penetration of pharmaceutical substances into or through the biological membranes and tissues, such as, but not limited to, mucous membranes.

As used herein the term “oro-dispersible drug formulation” refers to structures that contain active pharmaceutical substances. Examples include, but are not limited to, organic molecules that are stable in the solid state, but rapidly disintegrate on the moist surface of mucous membranes or buccal mucosa that line the oral cavity and tongue. These disintegrates form solutions or suspensions of the active pharmaceutical substance(s) and are absorbed into the body without the need to swallow the active pharmaceutical substances and thereby preclude either gastric or intestinal absorption. Non-limiting examples are: oro-dispersible lyophilisates (ORL), oro-dispersible tablets (ODT), oro-dispersible granules (ODG), oro-dispersible films (ODF) and oro-dispersible fiber meshes (OFMs). In all oro-dispersible drug formulations, other substances may also be incorporated, such as, but not limited to; flavorings, taste masking substances, binders, volumizers, and penetration enhancers.

As used herein the term “Orodispersible lyophilisates” (ORLs) refers to solid dosage forms which are produced from active pharmaceutical substances in solutions or suspensions which are freeze-dried. Typically, there are also matrix forming excipients including, but not limited to; gelatin, dextran, alginates, glycine, or mannitol used to increase the physical volume of the ORL and prevent shrinking during freeze-drying.

As used herein the term “Orodispersible tablets (ODTs)” refers to tablets that dissolve or disintegrate in the mouth within 60 seconds or less without the addition of water when placed on or under the patient's tongue or the buccal tissue of the cheek. They may be formulated with additional substances to enhance mucosal permeability of the active pharmaceutical substance or to enable muco-adhesion of the ODT or to mask the taste of the active pharmaceutical substance. ODTs, as used herein may also be considered synonymous with the terms “Orally Disintegrating Tablet” or “Troche” or “Lozenge”.

As used herein the term “Orodispersible granule” (ODGs) refers to a multi-particulate dosage form in which a single or multiple active pharmaceutical substance(s) are composed of multiple small-sized dose carriers. Commonly, although not always, the carriers are manufactured by granulation techniques. They can be directly administered into the mouth of the subject patient or sprinkled on food before the oral administration. ODGs spread within the oral cavity after administration and dissolve to release the active pharmaceutical substance(s) to form a solution or suspension.

As used herein the term “Orodispersible films” (ODFs) refers to solid films or patches that are single or multilayer sheets of suitable material which rapidly disperse or dissolve when administered to the oral cavity/oral mucosa to release the active pharmaceutical substance(s) to form a solution or suspension.

As used herein the term “Orodispersible fiber meshes” (OFMs) refers to fibers or meshes or webs of fibers that are often but not always created by electrospinning various polymers that contain active pharmaceutical substance(s) where the fibrous material rapidly disperses or dissolves when administered to the oral cavity to release the active pharmaceutical substance(s) to form a solution or suspension.

As used herein the term “pre-gastric” refers to delivery and absorption of an active pharmaceutical composition that does not involve the active pharmaceutical composition coming into contact with the surface of either the gastric or intestinal mucosa. Therefore, it refers to the absorption of active pharmaceutical compositions that does not occur in the intra-abdominal part of the gastrointestinal (GI) tract. The intra-abdominal portion of the gastrointestinal tract begins at the diaphragmatic hiatus and ends at the anus.

As used herein the term “nasal delivery” (or nasal drug delivery) refers to a method of delivering or applying an active pharmaceutical composition to the nasal mucosa. Most commonly, but not exclusively, the active pharmaceutical composition is insufflated or inhaled through the nose. However, the active pharmaceutical composition can be directly applied to the mucosa of the nasal cavity by direct application of, but not limited to, a liquid, solid, semi-solid, gaseous, mist or vaporized form of the active pharmaceutical composition. A common method, but not the only method, is for the active pharmaceutical composition to be contained in a solution that can be sprayed to create an aerosol that can be insufflated or inhaled through the nose and deposited into the nasal cavity. The drug substance itself can be in natural form of a simple solution or an emulsion, microemulsion or nano-emulsion or be contained within a superstructure such as, without limitation; a nano-structured carrier, microcarrier, liposome, granule, or gel form.

As used herein the term “inhaled” refers to the act of taking a substance into the body by breathing in or inspiring. As used herein the term “inhaled” is synonymous with “inhalation.” Most commonly, but not always, the substance is in a vapor or mist containing droplets, an aerosol containing a particulate or droplets, or in a gaseous form.

As used herein the term “nano-structured carrier” refers to nano-sized compositions capable of being combined with active pharmaceutical compound to form a vehicle or device useful to deliver such pharmaceutical compositions to an animal subject. Generally, the International Organization for Standardization (ISO) defines a nanostructured material as a “material with any external nanoscale dimension or having internal nanoscale surface structure.” Nanoscale dimension (nano-sized) is defined as measuring from 1 to 100 nanometers. Organic nanostructures such as, but not limited to; dendrimers, liposomes, micelles, polymer nanoparticles, gels, etc., are made mostly from carbon based organic materials, excluding toxic carbon-based or inorganic-based nanostructures. These nanostructures are nontoxic, biodegradable, and some of their structures, e.g., liposomes and micelles, have hollow cores (also known as nano-capsules).

As used herein the terms “topical” or “topically applied” or “administered topically” are synonymous and refer to applying or depositing a pharmaceutically active compound or compounds on the surface of an epithelium or epithelial tissue such as, but not limited to, a mucosa membrane or tissue, or the skin (epidermis).

Molindone (3-ethyl-2-methyl-5-(morpholin-4-ylmethyl)-1,5,6,7-tetrahydroindol-4-one) is a drug compound originally and mainly used in schizophrenia treatment. In addition, its use has also been considered as an adjunctive therapy for subjects with ADHD (attention-deficit/hyperactivity disorder) who are also simultaneously using stimulant drugs such as, but not limited to, methylphenidate, amphetamine.

Molindone is primarily, if not exclusively, metabolized by the liver (hepatic metabolism). The first metabolite of molindone obtained by this liver metabolism is 3-ethyl-2-(hydroxymethyl)-5-[(morpholin-4-yl)methyl]-1,5,6,7tetrahydro-4H-indol-4-one. Therefore, molindone use in treatment needs to be monitored due to possible hepatotoxic impact. This entails periodic monitoring of hepatic function by blood tests measuring biomarkers of hepatotoxicity. Drug substances absorbed from the stomach or intestine after ingestion travel via the portal vein first to the liver where a large fraction of the dose administered is initially metabolized to derivative compounds in a process termed “first pass metabolism”. First pass metabolism reduces the amount of the actual biologically active form of drugs such as, but not limited to, molindone that are then carried to the rest of the organs of the body by circulation in the blood. First pass metabolism by the liver in drugs such as, but not limited to, molindone also results in drug metabolites which are potentially toxic, and which often result in undesirable side effects. This is termed the “first pass effect”.

Herein the inventors disclose that they have unexpectedly discovered that Molindone administered either as a racemate or as a specific enantiomer are very effective in reducing the aberrant behavior and motor symptoms caused by autism and autism spectrum disorders as well as several other psycho-social symptoms caused by the condition.

We have also found that administration using a pre-gastric direct trans-mucosal route via topical application to buccal, lingual, sublingual, nasal or respiratory mucosal achieves the maximal plasma concentration of the drug faster than when given via the stomach and/or intestines and also achieves a higher total plasma level due to by-passing the first pass metabolism in the liver. This results in improved bioavailability, reduces absorptive variability of molindone based on food intake, reduces the dose required for treatment, and achieves a more rapid onset of action. Levels of potentially problematic toxic metabolites due to the firstpass effect are reduced as well.

In addition, we have surprisingly discovered that the dextrorotatory form of molindone has novel physiochemical properties from the levorotary form and is not associated with the certain specific side effect seen in the racemate and levorotary form itself.

In one aspect, the molindone is administered topically to the buccal, lingual, or sublingual mucosa of a subject in a dose form that does not require swallowing a dose form such, as but not limited to, a tablet or a liquid dose form. Here, the absorption of the molindone is trans-mucosal and does not require passage into the liver, stomach, or intestines.

In another aspect, the molindone may be administered topically to the buccal, lingual, or sublingual mucosa of a subject in a dose form that is oro-dispersive so as to facilitate dissolution of the formulation onto the surface of the mucosal epithelium.

In yet another aspect, the molindone is administered topically to the nasal or respiratory mucosa using devices and formulations such as, but not limited to, nasal sprays containing physiological buffers and other conventional excipients, well known in the art such as, but not limited to; water, sodium chloride, dextrose, polysorbate 80, carboxymethylcellulose sodium, benzalkonium chloride or phosphate buffered saline in various fixed proportions to achieve an isotonic solution and enable insufflation, inhalation and/or inspiration.

In in still another aspect, the molindone is prepared to include an ingredient or drug delivery technology that is muco-adherent such that attachment onto the surface of the mucosal tissue is achieved. Various substances are known to those skilled in the art, such as but not limited to; chitosan, xanthum gums, sodium carboxymethylcelluose, hydroxypropylmethylcelluose, Carbopol, thiolated hyaluronic acid and polylactic acid. These and other chemical compounds with mucoadhesive properties can also be incorporated into nano-structured carriers that also contain molindone to facilitate attachment to the mucosal tissue.

In another aspect, the molindone is incorporated into a nano-structured carrier device to facilitate administration. The nano-structured carrier device that contains the molindone can be muco-adherent for the purpose of drug delivery. A non-limiting example is the incorporation by methods known to those skilled in the art of a therapeutic amount of molindone into a nanoparticle composed of chitosan or polylactic acid. This and other exemplar nano-structured carrier devices can be used to delivermolindone to various mucosal tissues.

In one aspect, the treatment is affected without causing weight gain and/or having lowered levels of primary metabolites of molindone in the subject. In another aspect, the molindone is a salt of the (+) enantiomer of molindone. In still another aspect, the molindone or salt thereof is a racemate. The effective amount of molindone will depend on the body weight of the subject but can be between about 0.2 mg to 20 mg per kg of body weight. In yet another aspect, a clinician skilled in the art will administer the molindone in a fashion consistent with good medical practice and determine the exact dosage according to the symptoms of the patient under treatment.

In some aspects, the effective amount of molindone is initiated at about 0.2 mg/kg of body weight daily and titrated up to a total dose of about 40 mg daily. The effective dose will be determined by the clinician treating the subject or patient. As a non-limiting example, the amount of molindone can also be initiated at about 5 mg daily and titrated up to higher doses as needed to effectively reduce or eliminate the symptoms over a period of time. In some aspects, the effective amount of molindone can be administered in a single daily dose or in two or three daily divided doses as may be done by those skilled in the art. In the case of the dextrorotary form, the dose can be reduced to less than the equivalent effective dose of the racemate. For example, it can be expected that an effective dose of the dextrorotatory form of molindone alone could be from 30 to 70 percent of the effective dose of the racemate.

In one aspect, molindone can be formulated in a fast-melt tablet oral disintegrating tablet, or mucoadhesive gel, or in a mucoadhesive nanoparticle carrier. In some aspects, molindone is formulated as a permeation-enhanced gel or nanoparticle. The gel can contain between 0.5% (wt./wt.) molindone to 5% (wt./wt.) molindone without other excipients. In some aspects, the gel contains 0.5% (wt./wt.) molindone to 5% (wt./wt.) molindone with other excipients well known to those skilled in the art to facilitate suitable topical formulation either for the epidermis or mucosal tissues.

In one aspect, alleviating one or more behavioral or motor symptoms of ASD can include an improvement in a total score of an instrument measuring a range of aberrant behaviors in a subject. In another aspect, a single aberrant behavior is alleviated such as, but not limited to, irritability or social avoidance of the subject. In some aspects, alleviating one or more behavioral symptoms of ASD can include improvement in one or more measures of an Aberrant Behavior Checklist (ABC).

In some aspects, the one or more aberrant behavior symptoms present are selected from the group consisting of irrational or exaggerated fear, social avoidance, compulsive behavior, manic behavior, irritability, lethargy, stereotypy, inappropriate speech and any combination(s) thereof, that can be quantitated by a suitable psychometric instrument such as, but not limited to, the Aberrant Behavior Checklist (ABC).

Administering an effective amount of molindone in the forms and methods described herein can reduce the risk or intensity of at least one adverse event or side effect relative to administering molindone via gastric or intestinal absorption. in one aspect, the at least one adverse event or side effect can be a weight gain. In some aspects, the at least one adverse event is somnolence wherein the frequency and/or intensity of somnolence is reduced as an adverse event.

The following are non-limiting examples of the use of molindone in treatment of one or more motor and/or behavioral symptoms of autism spectrum disorder as well as treatment of those symptoms also found in subjects diagnosed with, for example, Fragile X, tuberous sclerosis, Smith-Lemli-Opitz syndrome, and maternally-inherited duplications of the Prader-Willi/Angelman syndrome region, unexpected properties of the dextrorotary isomer, and the unexpected pharmacologic findings using the pre-gastric dose route.

### EXAMPLE 1

In an open-label observational trial by an investigator, 11 patients with ASD were treated for eight weeks with molindone starting at 5 mg/day and the dosage increased by the patient and caregiver in 5 mg increments as tolerated by the subjects up to a maximum dose of 25 mg/day or until amelioration of the aberrant behaviors was seen. Patient response was scored using the ABC-C scale. The data in Table 1 demonstrate a significant improvement during the course of the study in reduction of symptoms with the use of molindone in all five categories, including stereotypy with all p values less than 0.05.

TABLE 1
ABC-C Scale Mean Scores N =11

| | | | Decrease | |
|---|---|---|---|---|
| Subscale | | | from | Significance |
| (scoring range) | Baseline | Week 8 | baseline (%) | (p < 0.05) |
| Irratability | 35.6 | 8.3 | 76.6 | <0.0001 |
| (0-45) | | | | |
| Stereotypy | 11.8 | 3.2 | 72.8 | 0.0081 |
| (0-21) | | | | |
| Hyperactivity/ | 29.8 | 11.2 | 62.4 | <0.0001 |
| Noncompliance | | | | |
| (0-48) | | | | |
| Inappropriate speech | 9.6 | 2.2 | 77 | 0.0048 |
| (0-12) | | | | |
| Lethargic/Withdrawn | 16.5 | 6.4 | 63.2 | 0.0069 |
| (0-48) | | | | |
| Total Score | 103.3 | 31.3 | 69.9 | 0.0001 |
| (0-174) | | | | |

Subjective responses from the caregivers of the subjects as well as several of the subjects themselves, indicated that during the course of therapy noticeable and desirable weight loss occurred to the subjects. Moreover, none complained of increased somnolence during the course of the study, indicating a reduction in frequently reported side effects.

### EXAMPLE 2

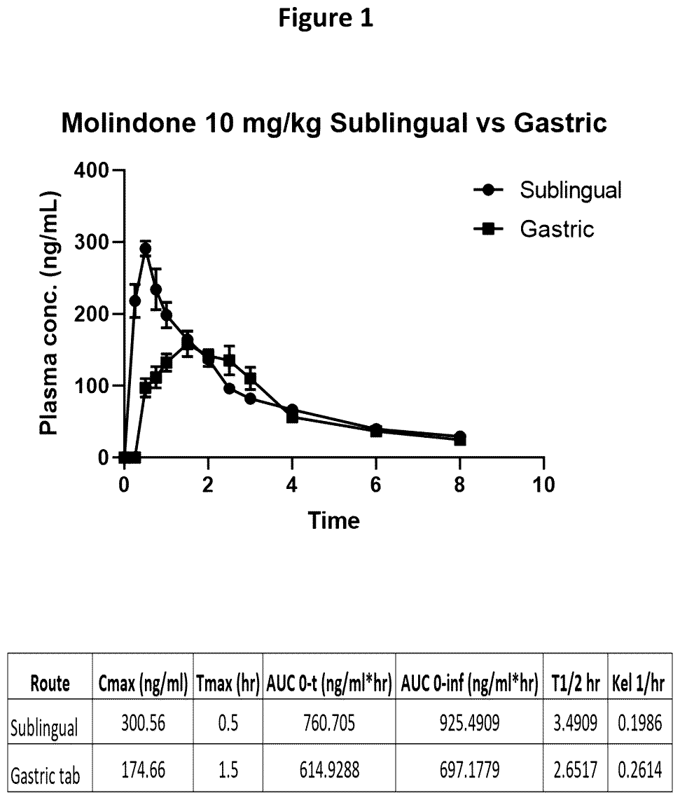

A study of equivalent doses of molindone administered by gastric absorption compared to sublingual absorption. Pharmacokinetic data demonstrate better bioavailability of the sublingual dosing compared to gastric dosing. Data in FIG. 1 show a higher Cmax (highest plasma concentration of the drug) obtained by sublingual dosing. The Tmax (time to achieve the Cmax) is shorter in the sublingual dosing compared to gastric dosing demonstrating a more rapid attainment of an effective plasma concentration of the molindone from sublingual dosing. The T1/2, which measures the time at which the drug has lost half of its maximum plasma concentration, is longer when molindone is administered in a non-gastric dosing regimen. In addition, the area under the curve (AUC), which is the definite integral of the concentration of a drug in blood plasma as a function of time, is better using sublingual dosing. Collectively, this study demonstrates better relative bioavailability of molindone when administered in a method that does not involve gastric drug delivery (i.e., pre-gastric delivery). Molindone is primarily, if not exclusively, metabolized by the liver (hepatic metabolism). The first metabolite of molindone obtained by liver metabolism is 3-ethyl-2-(hydroxymethyl)-5-[(morpholin-4-yl)methyl]-1,5,6,7tetrahydro-4H-indol-4-one. The level of this metabolite was also measured at the Cmax of the sublingual and gastric dosing regimens. No detectable metabolite was found at Cmax of the sublingual dosing route in contrast to 37.2 ng/mL being present at the Cmax of the gastric dosing. This indicates a reduction of metabolic side products occurs when pre-gastric dosing is used.

### References

Lord C, et al. The Lancet, Volume 392, Issue 10146, 508-520.
Bertrand J, et al. Pediatrics. 2001 November; 108(5):1155-61.
American Psychiatric Association. Diagnostic and Statistical Manual of Mental Disorders (DSM-V). 5th Edition. Washington, DC: American Psychiatric Association; 2013:
Yeargin et al. JAMA 2003, 289:49-55;
Aman M, et al. American Journal of Mental Deficiency 1985, 89:485-491.
Aman M, et al. The Aberrant Behavior Checklist-Community. East Aurora, NY: Slosson Education Publications, Inc.; 1994
Stocks J D K, et al. J. Child. Adolesc. Psychopharmacol. 2012, 22, 102-111.
Krishna G. et al. Environ. Mol. Mutagen. 2016, 57, 288-298.
Krishna G, et al. Toxicol. Mech. Methods 2017, 27, 352-362.

What is claimed is:

1. A method of treating a human subject suffering from autism spectrum disorder comprising topically administering to the subject's pre-gastric mucosa a therapeutically effective amount of a stable, immediate release molindone formulation and/or a pharmaceutically acceptable molindone salt formulation to reduce or eliminate one or more symptoms thereof, wherein the formulation(s) bypasses the first-pass effect.

2. A method of treating a human subject suffering from motor or behavior symptoms of autism spectrum disorder comprising topically administering to the subject's pre-gastric mucosa a therapeutically effective amount of a stable, immediate release molindone formulation and/or a pharmaceutically acceptable molindone salt formulation to reduce or eliminate one or more motor and/or behavioral symptoms thereof, wherein the formulation(s) bypasses the first-pass effect.

3. The method according to claim 1, wherein the human subject is diagnosed as having; Tuberous Sclerosis Complex, Fragile X, Cornelia de Lange, Down, Prader-Willi/Angelman, Coffin-Lowry, Cohen Laurence-Moon-Biedel, Cri-du-chat, Marinesco-Sjogren, Moebius, Rett, Smith-Lemli-Opitz, and/or Williams syndromes.

4. The method according to any one of claim 1, 2, or 3, wherein the treatment reduces or eliminates one or more motor symptoms.

5. The method according to any one of claim 1, 2, or 3, wherein the treatment reduces or eliminates one or more behavioral symptoms.

6. The method according to claim 1, wherein the molindone formulation and/or the pharmaceutically acceptable molindone salt formulation comprises the levorotatory and/or the dextrorotatory enantiomer.

7. The method of claim 6, wherein the molindone formulation and/or pharmaceutically acceptable molindone salt formulation comprises the dextrorotatory enantiomer of molindone in the absence of any substantial amount of the levorotatory enantiomer.

8. The method of claim 6, wherein the molindone formulation and/or pharmaceutically acceptable molindone salt formulation comprises the levorotatory enantiomer of molindone in the absence of any substantial amount of the dextrorotatory enantiomer.

9. The method of claim 1, wherein the molindone formulation and/or pharmaceutically acceptable molindone salt formulation is administered along with another pharmaceutical agent.

10. The method of claim 1, wherein the molindone formulation and/or pharmaceutically acceptable molindone salt formulation is administered topically to the oral, buccal, lingual, sublingual, nasal, and/or respiratory mucosa.

11. The method of claim 1, wherein the molindone formulation and/or pharmaceutically acceptable molindone salt formulation is administered with a mucosal penetration enhancer.

12. The method of claim 1, wherein the molindone formulation and/or pharmaceutically acceptable molindone salt formulation is administered in a topically applied nano-structured carrier.

13. The method of claim 12, wherein the nanostructured carrier has muco-adherent properties.

14. The method of claim 1, wherein molindone formulation and/or pharmaceutically acceptable molindone salt formulation is administered in a topically applied oro-dispersible formulation.

15. The method of claim 1, wherein molindone formulation and/or pharmaceutically acceptable molindone salt formulation is administered in a topically applied oro-dispersible film formulation.

16. The method of claim 15, wherein the oro-dispersible film formulation has mucosal adherent properties.

17. The method of claim 1, wherein the molindone formulation and/or pharmaceutically acceptable molindone salt formulation is administered in a topically applied oro-dispersible tablet that disintegrates directly on the oral mucosal surface in 60 seconds or less without addition of water.

18. The method of claim 1, wherein the molindone formulation and/or pharmaceutically acceptable molindone salt formulation is administered in a topically applied oro-dispersible granule formulation.

19. The method of claim 18, wherein the oro-dispersible granule formulation has mucosal adherent properties.

20. The method of claim 1, wherein the molindone formulation and/or pharmaceutically acceptable molindone salt formulation is administered in a topically applied oro-dispersible lyophilisate that dissolves or disintegrates directly on the mucosal surface.

21. The method of claim 1, wherein the molindone formulation and/or pharmaceutically acceptable molindone salt formulation is administered in a topically applied oro-dispersible fiber formulation.

22. The method of claim 21 wherein the oro-dispersible fiber formulation has mucosal adherent properties.

23. The method of claim 1, wherein the molindone formulation and/or pharmaceutically acceptable molindone salt formulation is administered topically via nasal delivery to the nasal mucosa by insufflation or inhalation.

24. The method of claim 1, wherein molindone is administered at least once per day.

25. A method of treating a human subject with an Aberrant Behavioral Checklist score equal to or greater than the 50^{th} percentile or a T-score equal to or greater than 50 comprising topically administering to the human subject's pre-gastric mucosa a therapeutically effective amount of a stable, immediate release molindone formulation and/or a pharmaceutically acceptable molindone salt formulation to reduce or eliminate one or more motor and/or behavioral symptoms thereof, wherein the formulation(s) bypasses the first-pass effect and the Aberrant Behavioral Checklist score is the score for the categories of irritability, stereotypy, hyperactivity/noncompliance, inappropriate speech, and lethargic/withdrawn.
